(19) United States
(12) Patent Application Publication
(10) Pub. No.: US 2016/0128294 A1
(43) Pub. Date: May 12, 2016

(54) INFUSED PLANT SEEDS

(71) Applicant: R.J. REYNOLDS TOBACCO COMPANY, Winston-Salem, NC (US)

(72) Inventors: Brian Keith Nordskog, Winston-Salem, NC (US); Darlene Madeline Lawson, Kernersville, NC (US)

(21) Appl. No.: 14/537,181

(22) Filed: Nov. 10, 2014

(51) Int. Cl. A01H 5/12; A01C 1/06

(52) U.S. Cl. CPC A01H 5/12; A01C 1/06

(57) ABSTRACT

Treated plant seeds are provided, which have one or more modifying ingredients associated therewith. Such modifying ingredients can include compositions comprising water, flavorants, pH adjusters, buffering agents, humectants, antioxidants, oral care ingredients, preservatives, additives derived from herbal or botanical sources, and mixtures thereof that can be provided or diluted form. The treated plant seeds can be incorporated within tobacco products, including smoking articles, to alter the properties thereof (e.g., by releasing the one or more modifying ingredients into the mainstream smoke of a smoking article).

### FIELD OF THE INVENTION

The present invention relates to products made or derived from tobacco, or that otherwise incorporate tobacco, and are intended for human consumption.

### BACKGROUND OF THE INVENTION

Cigarettes, cigars and pipes are popular smoking articles that employ tobacco in various forms. Such smoking articles are used by heating or burning tobacco, and aerosol (e.g., smoke) is inhaled by the smoker.

The sensory attributes of cigarette smoke can be enhanced by applying various materials to tobacco and/or by otherwise incorporating flavoring materials into various components of a cigarette. See, Leffingwell et al., Tobacco Flavoring for Smoking Products, R.J. Reynolds Tobacco Company (1972). For example, one type of tobacco flavoring additive is menthol. See, Borschke, Rec. Adv, Tob. Sci., 19, p. 47-70, 1993. Various proposed methods for modifying the sensory attributes of cigarettes have employed filter elements as vehicles for adding flavor to the mainstream smoke of those cigarettes. Representative types of cigarette filters incorporating flavoring agents are set forth in U.S. Pat. No. 3,972,335 to Tiggelbeck et al.; U.S. Pat. No. 4,082,098 to Owens, Jr.; U.S. Pat. No. 4,281,671 to Byrne; U.S. Pat. No. 4,729,391 to Woods et al.; U.S. Pat. No. 4,862,905 to Green, Jr. et al.; U.S. Pat. No. 4,941,486 to Dube et al.; U.S. Pat. No. 4,991,596 to Lawrence et al.; and U.S. Pat. No. 5,012,829 to Thesing et al., which are incorporated herein by reference.

Cigarettes having adjustable filter elements that allow smokers to select the level of flavor that is available for transfer into mainstream smoke have been proposed. See, for example, U.S. Pat. No. 4,677,995 to Kallianos et al. and U.S. Pat. No. 4,848,375 to Patron et al., which are incorporated herein by reference. Some proposed cigarettes may be manipulated, reportedly for the purpose of providing components of their filter elements with the propensity to modify the nature or character of mainstream smoke. See, for example, U.S. Pat. No. 3,297,038 to Homburger; U.S. Pat. No. 3,339,557 to Karalus; U.S. Pat. No. 3,420,242 to Boukar; U.S. Pat. No. 3,508,558 to Seyburn; U.S. Pat. No. 3,513,859 to Carty; U.S. Pat. No. 3,596,665 to Kindgard; U.S. Pat. No. 3,669,128 to Cohen; and U.S. Pat. No. 4,126,141 to Grossman, which are incorporated herein by reference.

Some proposed cigarettes have objects (e.g., pellets, beads, or breakable capsules) positioned in their filter element, and the contents of such objects can reportedly be released into the filter element upon rupture of the object in the attempt to alter the nature or character of the mainstream smoke passing through the filter element. See, for example, the types of technologies set forth in U.S. Pat. No. 3,339,558 to Waterbury; U.S. Pat. No. 3,366,121 to Carty; U.S. Pat. No. 3,390,686 to Irby, Jr. et al.; U.S. Pat. No. 3,428,049 to Leake; U.S. Pat. No. 3,547,130 to Harlow et al; U.S. Pat. No. 3,575,180 to Carty; U.S. Pat. No. 3,602,231 to Dock; U.S. Pat. No. 3,625,228 to Dock; U.S. Pat. No. 3,635,226 to Horsewell et al.; U.S. Pat. No. 3,685,521 to Dock; U.S. Pat. No. 3,916,914 to Brooks et al.; U.S. Pat. No. 3,991,773 to Walker; U.S. Pat. No. 4,889,144 to Tateno et al.; U.S. Pat. No. 6,631,722 to MacAdam et al. U.S. Pat. No. 7,115,085 to Deal; U.S. Pat. No. 7,479,098 to Thomas et al.; U.S. Pat. No. 7,833,146 to Deal; U.S. Pat. No. 7,972,254 to Stokes et al.; U.S. Pat. No. 7,984,719 to Dube et al.; U.S. Pat. No. 8,066,011 to Clark et al.; U.S. Pat. No. 8,262,550 to Barnes et al.; U.S. Pat. No. 8,353,810 to Garthaffner et al.; U.S. Pat. No. 8,381,947 to Garthaffner et al. and U.S. Pat. No. 8,459,272 to Karles et al.; U.S. Pat. No. 8,470,215 to Zhang; U.S. Pat. No. 8,512,213 to Deal, U.S. Pat. No. 8,622,882 to Nikolov et al.; U.S. Pat. No. 8,671,951 to Ercelebi et al.; U.S. Pat. No. 8,739,802 to Fagg; and U.S. Pat. No. 8,820,331 to Hartmann et al.; US Pat. Application Pub. Nos. 2002/0166563 to Jupe et al.; 2006/0112964 to Jupe et al.; 2006/0144412 to Mishra et al.; 2006/0174901 to Karles et al.; 2007/0012327 to Karles et al.; 2007/0095357 to Besso et al.; 2010/0184576 to Prestia et al.; 2011/0053745 to Iliev et al.; 2012/0245007 to Henley et al.; 2012/0255569 to Beard et al.; 2014/0053855 to Hartmann et al.; and WO 03/009711 to Kim and WO 2007/060543 to Besso et al., which are all incorporated herein by reference. Representative cigarette products that possess filter elements incorporating breakable capsules have been marketed throughout the world under the brand names such as, for example, “Marlboro W-Burst 5,” “Camel Crush,” “Kent iSwitch,” and “Kool Boost.” Smokeless tobacco products containing encapsulated ingredients have also been proposed, such as in U.S. Pat. No. 7,810,507 to Dube et al., which is incorporated herein by reference.

It would be desirable to provide further methods for modifying the sensory attributes of tobacco products.

### SUMMARY OF THE INVENTION

The present invention relates to a method for treating a plant seed to incorporate a modifying ingredient therein. The present invention further relates to the incorporation and use of such plant seeds within tobacco materials (e.g., smoking articles), wherein the modifying ingredient incorporated therein can be released from the plant seed during use of the tobacco materials.

In one aspect of the disclosure is provided a tobacco product comprising a tobacco formulation and at least one treated plant seed or portion thereof carrying one or more modifying ingredients. The treated plant seed can comprise an edible seed or portion thereof. For example, in various embodiments, the treated plant seed can comprise a seed selected from the group consisting of tobacco seeds, poppy seeds, sesame seeds, sunflower seeds, pumpkin seeds, chia seeds, flax seeds, hemp seeds, papaya seeds, cocoa seeds, and soybean seeds In one embodiment, the treated plant seed comprises a seed or portion thereof from the Nicotiana species.

The modifying ingredient(s) can vary and, in certain embodiments, are selected from the group consisting of water, flavorants, sweeteners, colorants, pH adjusters, buffering agents, oral care additives, humectants, antioxidants, preservatives, additives derived from herbal or botanical sources, and mixtures thereof. In some embodiments, the modifying ingredient comprises a flavorant, such as a flavorant selected from the group consisting of vanilla, coffee, chocolate, cream, mint, spearmint, eucalyptus, menthol, peppermint, wintergreen, lavender, cardamom, nutmeg, cinnamon, clove, cascarilla, sandalwood, honey, jasmine, ginger, anise, sage, licorice, lemon, orange, apple, peach, lime, cherry, strawberry, and combinations thereof. One exemplary flavorant is menthol.

Although in some embodiments, the treated plant seed or portion thereof carries only the one or more modifying ingredients, in other embodiments, the treated plant seed or portion thereof can carry one or more additional components. For example, in some embodiments, the treated plant seed or portion thereof may further comprise one or more diluting agents associated therewith. In certain embodiments, the diluting agent associated therewith comprises triglycerides.

The tobacco product comprising the at least one treated plant seed or portion thereof may be any type of tobacco product, including smoking articles and smokeless tobacco products. In smoking articles, the one or more modifying ingredients can be adapted for release into mainstream smoke generated by the smoking article. The treated plant seed or portion thereof can be incorporated, for example, within a filter element of a smoking article comprising a filter element. In some embodiments, a filter element can comprise at least one cavity comprising the at least one treated plant seed or portion thereof. In smokeless tobacco products, the one or more modifying ingredients can be adapted for release in the oral cavity.

In another aspect of the invention is provided a method for modifying the properties of a tobacco product, comprising: i) receiving a treated plant seed or portion thereof that has been subjected to a process whereby a modifying ingredient has become associated therewith; and ii) adding the treated plant seed to a tobacco product. In some embodiments, the method can further comprise treating a harvested plant seed or portion thereof by contacting the plant seed or portion thereof with a modifying ingredient to give a treated plant seed or portion thereof.

In one specific aspect, the invention provides a method for modifying the properties of a tobacco product, comprising: i) treating a harvested plant seed or portion thereof by contacting the plant seed or portion thereof with a modifying ingredient to give a treated plant seed or portion thereof; and ii) adding the treated plant seed or portion thereof to a tobacco product.

For example, the treating step can comprise immersing the harvested plant seed or portion thereof in a liquid comprising the modifying ingredient. The liquid comprising the modifying ingredient can, optionally, further comprise one or more solvents. The treating step may be conducted under various conditions (e.g., at room temperature or elevated temperature and/or at atmospheric pressure or elevated temperature). In certain embodiments, the method can further comprise the step of pre-treating the harvested plant seed or portion thereof having a seed coat to soften the seed coat or to create microfractures in the seed coat in order to enhance penetration of the modifying ingredient therethrough.

In the methods disclosed herein, the tobacco product may be in the form of a smoking article comprising a filter element, wherein the adding step comprises incorporating the treated plant seed or portion thereof within the filter element. In some embodiments, the filter element comprises at least one cavity and the adding step comprises incorporating the treated plant seed or portion thereof within the at least one cavity. For example, the treated plant seed or portion can be incorporated within the at least one cavity such that the cavity is substantially filled with the treated plant seed or portion thereof.

In one further aspect, the present disclosure provides a tobacco product in the form of a seed or portion thereof from the Nicotiana species carrying a modifying ingredient infused therein.

### BRIEF DESCRIPTION OF THE DRAWINGS

In order to provide an understanding of embodiments of the invention, reference is made to the appended drawings, which are not necessarily drawn to scale, and in which reference numerals refer to components of described exemplary embodiments of the invention. The drawings are exemplary only, and should not be construed as limiting the invention.

FIG. 1 is an exploded perspective view of a smoking article having the form of a cigarette, showing the smokable material, the wrapping material components, and the filter element of the cigarette;

FIG. 2 is a cross-sectional view of one embodiment of a filter element according to the present invention; and

FIG. 3 is a cross-sectional view of a smokeless tobacco product embodiment, taken across the width of the product, showing an outer pouch filled with a smokeless tobacco composition comprising treated plant seeds according to the present disclosure.

### DETAILED DESCRIPTION OF THE PREFERRED EMBODIMENTS

The present inventions now will be described more fully hereinafter with reference to the accompanying drawing. The inventions may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural referents unless the context clearly dictates otherwise.

The disclosure generally provides harvested plant seeds or portions thereof treated to associate one or more modifying ingredients therewith (e.g., by infusing the one or more modifying ingredients within the plant seed). In particular, the disclosure provides such plant seeds that can be incorporated within a tobacco product such that the modifying ingredient(s) can be released during use. For example, in some embodiments, such plant seeds can be incorporated within a smoking article such that the modifying ingredient can be released from the seed into the mainstream smoke and delivered to the user to provide the benefits associated with the modifying ingredient(s) to the user.

The principles described herein can be generally applied to any plant seed. A plant seed is understood to be an object produced by a plant from which a new plant can grow (i.e., the reproductive structure of a plant). Seeds typically consist of a plant embryo, optionally accompanied by a supply of food, and enclosed in a protective coat (e.g., seed coat). Generally, plant seeds useful according to the present disclosure include edible plant seeds, although useful plant seeds are not limited thereto. Edible plant seeds can include nuts, kernels, legumes, beans, and the like. Certain exemplary plant seeds that can be treated according to the methods described herein include, but are not limited to, tobacco seeds, poppy seeds, sesame seeds, sunflower seeds, pumpkin seeds, chia seeds, flax seeds, hemp seeds, papaya seeds, cocoa seeds, and soybean seeds. The remainder of the disclosure will be described with specific reference to tobacco seeds (i.e., seeds of a plant of the Nicotiana species); however, it is to be understood that the disclosure applies to other types of seeds that can be treated in a similar fashion.

In certain embodiments, it may be beneficial for the plant seed to be substantially round in shape. Round seeds can beneficially provide for good flowability and can be easily manipulated for use in various products. However, the disclosure is not limited thereto, and it is understood that seeds of a wide range of sizes and shapes can be treated as described herein (e.g., bean-shaped, disc-like, oblong, ovate, etc.). Furthermore, although substantially whole (unbroken) seeds are advantageously employed according to the present disclosure, seeds can also be employed in other forms. For example, seeds can be physically processed to subdivide the seeds into parts or pieces (e.g., the seed can be comminuted, pulverized, milled or ground into pieces or parts that can be characterized as granules, particulates or fine powders). “Whole” seeds as used herein is intended to refer to seeds that are complete and unbroken, as well as partial seed pieces and combinations thereof. Typically, such whole seeds are not defatted, compressed, or fractured internally and generally do not have substantial unnatural fissures or cracks therein.

With specific regard to seeds of the Nicotiana species, the seed is provided from the seed of the plant of the Nicotiana species, which is the characteristic reproductive structure of the plant (e.g., seed producing structure). See, for example, Frega et al., JAOCS, 68, 29-33 (1991); Patel et al., Tob. Res., 24, 44-49 (1998); Giannelos et al., Ind. Crops Prod., 16, 1-9 (2002); Mukhtar et al., Chinese J. Chem., 25, 705-708 (2007); Stanisavljevic et al., Eur. J. Lipid Sci. Technol., 111, 513-518 (2009); which are incorporated herein by reference. Tobacco seeds are used for various purposes, as disclosed, for example, in US Pat. Appl. Pub. No. 2011/0259353 to Coleman, I I I et al. and U.S. patent application Ser. No. 13/908,684 to Gerardi et al., filed Jun. 3, 2013, the contents of which are hereby incorporated by reference.

The selection of the plant from the Nicotiana species can vary; and in particular, the types of tobacco or tobaccos may vary. Tobaccos that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos. Descriptions of various types of tobaccos, growing practices and harvesting practices are set forth in Tobacco Production, Chemistry and Technology, Davis et al. (Eds.) (1999), which is incorporated herein by reference. Nicotiana species can be derived using genetic-modification or crossbreeding techniques (e.g., tobacco plants can be genetically engineered or crossbred to increase or decrease production of or to other change certain components, characteristics or attributes). For example, the Nicotiana species can be selected on the basis of producing relatively numerous seeds, or producing seeds that incorporate relatively high levels of specific desired components, and the like. Additional information on types of Nicotiana species suitable for use in the present invention can be found in US Pat. Appl. Pub. No. 2012/0192880 to Dube et al., which is incorporated by reference herein. The Nicotiana species of plant can be grown under agronomic conditions so as to promote seed and seed development. Tobacco plants can be grown in greenhouses, growth chambers, or outdoors in fields, or grown hydroponically.

The seed is harvested from the Nicotiana species of plant, and the manner by which the seed is harvested can vary. The seed can be removed from the rest of the plant by cutting or breaking the so-called seed head or seed capsule from the rest of the plant. Typically, virtually all of the seed (e.g., the whole seed) can be harvested, and employed as such. The various seeds can be isolated using typical mechanical separation and collection techniques. The time of harvesting the seed during the life cycle of the plant can vary. For example, the seed can be harvested when immature, and as such, the inflorescence or flower head can be removed from the plant. Alternatively, the seed head or seed capsule can be harvested from the plant after the point that the seed has reached maturity.

The post-harvest processing of the seed can vary. After harvest, the seed, or portion thereof, can be used in the harvested form (e.g., the seed can be used without being subjected to any curing and/or aging process steps). For example, the seed can be used without being subjected to significant storage, handling or processing conditions. In certain situations, it is preferable that the fresh seed be used virtually immediately after harvest. Alternatively, for example, seed can be refrigerated or frozen for later use, freeze dried, subjected to irradiation, yellowed, dried, cured (e.g., using air drying techniques or techniques that employ application of heat), heated or cooked (e.g., roasted, fried or boiled), or otherwise subjected to storage or treatment for later use. In some embodiments, the makeup of the seeds can be modified, e.g., by removing one or more components therefrom before treatment according to the present disclosure.

According to the present disclosure, the plant seed is subjected to treatment to associate one or more modifying ingredients therewith (e.g., by infusion of the modifying ingredient(s) into the seed). Attempts at infusion of various materials into certain types of plant seeds have been reported. For example, soybean seeds have been treated with fungicides with the goal of providing plant disease control. See Shortt et al., Disease Control and Pest Management, 70(10): 971-973 (1980) and Locke et al., Plant Disease, 67(9), 974-977 (September 1983). Plant seeds have been treated with bioactive chemicals (e.g., growth regulators and protectants) with the goal of alleviating certain environmental stresses (e.g., drought, supraoptimal temperatures, salinity, diseases, insect infestation, and soil pollution). See, e.g., Khan, Acta Hor, (ISHS) 83:225-234 (1978) and Khan et al., Plant Physiol. 52, 79-81 (1973). Barley seeds have been treated with hydrogen peroxide with the goal of modifying the germination process. See Cavusoglu et al., EurAsia J. BioSci. 4: 70-79 (2010). Soybean seeds have been treated with a hormone (kinetin) with the goal of reducing seed deterioration during storage. See Marwanto, Jurnal Ilmu-Ilmu Pertanian Indonesia 7(1):1-7 (2005). Certain roasted seeds (e.g., nuts) have been treated with additives such as salt, flavorings, nutrients, and colors to infuse such additives internally. See U.S. Pat. No. 8,435,579 to Smyth et al., which is incorporated herein by reference. Methods for treating plant seeds as described in these references may, in some embodiments, be applicable for the incorporation of modifying ingredients within plant seeds as described herein.

According to the present disclosure, a plant seed treated to associate one or more modifying ingredients therewith to provide a treated seed component can be added to a tobacco product (e.g., a smokable or smokeless tobacco product). Such “modifying ingredients” thus include any material that may be desirably associated with a tobacco product. For example, the modifying ingredient can comprise one or more components designed to alter the chemistry or sensory properties (e.g., flavor or aroma) of the tobacco material, or in the case of smokable tobacco materials, to alter the chemistry or sensory properties of mainstream smoke generated by smoking articles including the tobacco material. Typically, modifying ingredients of particular use according to the present disclosure are those that can be released from a treated plant seed, such as released into the mainstream smoke of a cigarette or released within the oral cavity in the case of a smokeless tobacco product. Exemplary modifying ingredients include, but are not limited to, water, flavorants, sweeteners, colorants, pH adjusters, buffering agents, oral care additives, humectants, antioxidants, preservatives, additives derived from herbal or botanical sources, or mixtures thereof. Exemplary plant-derived compositions that may be used are disclosed in US App. Pub. Nos. 2012/0152265 and 2012/0192880 to Dube et al, which are incorporated herein by reference. The selection of such modifying ingredients can vary based upon factors such as the sensory characteristics that are desired for the product, and the present invention is intended to encompass any such further components that may be readily apparent to those skilled in the art of tobacco and tobacco-related or tobacco-derived products. See, Gutcho, Tobacco Flavoring Substances and Methods, Noyes Data Corp. (1972) and Leffingwell et al., Tobacco Flavoring for Smoking Products (1972).

As used herein, a “flavorant” or “flavoring agent” is any flavorful or aromatic substance capable of altering the sensory characteristics associated with a tobacco composition. Exemplary sensory characteristics that can be modified by the flavorant include, taste, mouthfeel, moistness, coolness/heat, and/or fragrance/aroma. Types of flavorants include salts (e.g., sodium chloride, potassium chloride, sodium citrate, potassium citrate, sodium acetate, potassium acetate, and the like), natural sweeteners (e.g., fructose, sucrose, glucose, maltose, mannose, galactose, lactose, and the like), artificial sweeteners (e.g., sucralose, saccharin, aspartame, acesulfame K, neotame, and the like); and mixtures thereof. The flavorants can be natural or synthetic, and the character of these flavors can be described as, without limitation, fresh, sweet, herbal, confectionary, floral, fruity or spice. Exemplary flavorants include menthol and products of Maillard reactions, such as pyrazines, aminosugars, and Amadori compounds. Further specific types of flavors include, but are not limited to, vanilla, coffee, chocolate, cream, mint (e.g., mint oil), spearmint, menthol, peppermint, wintergreen, lavender, cardamon, nutmeg, cinnamon, clove, cascarilla, sandalwood, honey, jasmine, ginger, anise, sage, licorice, lemon, orange, apple, peach, lime, cherry, and strawberry. Flavorants utilized in the disclosure also can include components that are considered moistening, cooling or smoothening agents, such as eucalyptus. These flavors can be provided neat (i.e., alone) or in a composite (e.g., spearmint and menthol or orange and cinnamon). In one embodiment, the flavorant is menthol.

In some embodiments, the modifying ingredients may be throat irritation mitigants, including, but not limited to, sodium citrate, honey, ginger, pectin, capsaicin, camphor, dextromethorphan, echinacea, zinc gluconate, peppermint oil, spearmint oil, eucalyptus oil, glycerin, organic acids, and combinations or extracts thereof. In some embodiments, the modifying ingredients may be pH adjusters or buffering agents (e.g., metal hydroxides, preferably alkali metal hydroxides such as sodium hydroxide and potassium hydroxide, and other alkali metal buffers such as metal carbonates, preferably potassium carbonate or sodium carbonate, or metal bicarbonates such as sodium bicarbonate, and the like), humectants (e.g., glycerin, propylene glycol, and the like), oral care additives, preservatives (e.g., potassium sorbate, and the like), or syrups (e.g., honey, high fructose corn syrup, and the like used as flavorants). Other types of modifying ingredients that may be incorporated in some embodiments are set forth in U.S. Pat. No. 5,387,416 to White et al. and U.S. Pat. No. 7,984,719 to Dube et al.; U.S. Pat. App. Pub, No. 2005/0244521 to Strickland et al.; and WO 05/041699 to Quinter et al., each of which is incorporated herein by reference.

The method by which the modifying ingredient is associated with the plant seed according to the present disclosure can vary. The plant seeds can, in some embodiments, be treated by soaking the seeds in the modifying ingredient or by otherwise applying the modifying ingredient (e.g., by tumbling seeds in a rotating or agitating apparatus such that the modifying ingredient infuses into the seeds). Any means by which a liquid can be associated with a plant seed is intended to be encompassed according to the present application, which would allow for one or more modifying ingredients to become associated with (e.g., infused into) a plant seed.

In one embodiment, plant seeds are treated by incubating the seeds with the modifying ingredient. In such embodiments, the modifying ingredient may be provided in neat form or can be dissolved, suspended, or dispersed in one or more diluting agents (e.g., solvents) during the incubation step. Such diluting agents can vary and may be organic or aqueous. One preferred diluting agent is a triglyceride, such as a medium chain triglyceride (MTC), and more particularly a food grade mixture of medium chain triglycerides. See, for example, Radzuan et al., Porim Bulletin, 39, 33-38 (1999). It is noted that, in some embodiments, the diluting agent may become associated with the treated plant seed and, in other embodiments, the diluting agent does not become associated with the treated plant seed. Accordingly, in some instances, a treated seed is provided wherein substantially the entire material associated with (e.g., infused within) the treated seed consists of the modifying ingredient. In some instances, a treated seed is provided wherein at least a part of the material associated with (e.g., infused within) the treated seed comprises a diluting agent (e.g., the material associated with the treated seed can be almost entirely comprised of diluting agent, and only contain a very small amount of (generally relatively potent) modifying ingredient). Within a given treated seed, the composition of the mixture of modifying ingredient and diluting agent is in the range of about 5 percent to about 100 percent modifying ingredient, with certain embodiments being in the range of about 5 to about 90 percent modifying ingredient (balance diluting agent), and most preferably in the range of about 10 to about 25 percent modifying ingredient (balance diluting agent) by weight. The exact amount of modifying ingredient will depend on several factors including the modifying ingredient type and the desired sensory profile of the product.

The conditions under which the incubation is conducted (e.g., time, temperature, pressure, etc.) can vary. It is noted that the conditions may depend, in part, on the specific type of seed, the specific form of that seed (e.g., whole, broken, expanded, etc.), and/or the specific modifying ingredients to be infused therein. In some embodiments, the incubation is conducted at room temperature. In other embodiments, the incubation is conducted at elevated temperature (e.g., greater than room temperature, e.g., above about 25° C., above about 30° C., or above about 35° C., such as between about 20° C. and about 50° C., or between about 25° C. and about 40° C.). The incubation is generally conducted at atmospheric pressure, although it may be beneficial in some embodiments to conduct the incubation at elevated pressure. For example, in certain embodiments, high pressure processing (“HPP”) can be employed to associate the one or more modifying ingredients with the seed, e.g., as described in Baldo et al., J. Food Sci. Eng. 2 (2012), 543-549 and Balasubramaniam et al., Food Sci, Tech. Eng. 14(5) (2008), 413-418, which are incorporated herein by reference.

In certain embodiments, pretreatment of the plant seeds can enhance penetration of the modifying ingredient(s) into the seeds. For example, in one embodiment, the harvested plant seeds can be presoaked to expand the seed coat prior to the incubation step. This step may be particularly beneficial where the seed to be treated comprises a hard seed coat. For example, in some embodiments, plant seeds may be first soaked in water (at room or elevated temperature) to soften the seed coat and expand the pores contained therein. Alternatively, in some embodiments, the plant seeds may be first subjected to water vapor at elevated temperature. See, for example, Intl. App. Pub. No. WO2004/066751 to Van Der et al., which is incorporated herein by reference. This pretreatment to soften the seed coat may allow for greater penetration of the modifying ingredient through the seed coat while the seed coat is in softened/expanded form. Accordingly, the plant seeds in softened/expanded form can be brought into contact with the modifying ingredient(s) and may in some embodiments exhibit enhanced uptake and/or retention of the modifying ingredient(s) as they contract.

Although in some embodiments, the plant seeds may be treated as disclosed above in “raw” form, it can be beneficial in some embodiments, to “cook” (e.g., bake or roast) the seeds. This cooking can be done prior to the incubation step, concurrently with the incubation step, or following the incubation step. Cooking can be done in continuous or batch systems, with heating taking place through direct contact with warm air or through indirect contact. Roasting is understood to mean heat treatment of the plant seed, wherein changes to the flavor, aroma, or color of the plant seeds can be observed. Roasting can also, in some embodiments, result in the formation of microfractures in the seed coat, which can be beneficial for the incorporation of the modifying ingredient(s), particularly where the roasting is conducted prior to treatment with the modifying ingredient(s). In such embodiments, the modifying ingredient(s) may more readily be able to penetrate through the seed coat by way of the microfractures.

The treated seeds described herein advantageously comprise some amount of the modifying ingredient(s) and, optionally, some amount of diluting agent. In preferred embodiments, the treated seeds are infused with some amount of modifying ingredient(s) and, optionally, diluting agent. The modifying ingredient (and, optionally, diluting agent) may be contained within the plant seed (including within the seed coat) and/or coated on the exterior of the plant seed. The amount of modifying ingredient and, optionally, diluting agent within the plant seed can vary.

In accordance with the present disclosure, the treated plant seeds described herein can be incorporated in some form within a tobacco product. That is, a portion of the tobacco product can be composed of some form of the treated seed, such as parts or pieces of the seed, or processed materials incorporating processed seed or components thereof. The amount of treated plant seed incorporated within a tobacco composition, or otherwise incorporated within a tobacco product, can depend on the desired function of that treated plant seed (which can depend, at least in part, on the makeup of the modifying ingredient), the chemical makeup of the treated plant seed, and the overall type of tobacco composition into which the treated plant seed is incorporated.

The treated plant seed can be employed within a tobacco product in a variety of forms. The treated plant seed can be employed as a component of processed tobaccos. In one regard, the treated plant seed, or components thereof, can be employed within a top dressing formulation, or within a casing formulation for application to tobacco strip (e.g., using the types of manners and methods set forth in U.S. Pat. No. 4,819,668 to Shelar, which is incorporated herein by reference). Alternatively, the treated plant seed, or components thereof, can be employed as an ingredient of a reconstituted tobacco material (e.g., using the types of tobacco reconstitution processes generally set forth in U.S. Pat. No. 5,143,097 to Sohn; U.S. Pat. No. 5,159,942 to Brinkley et al.; U.S. Pat. No. 5,598,868 to Jakob; U.S. Pat. No. 5,715,844 to Young; U.S. Pat. No. 5,724,998 to Gellatly; and U.S. Pat. No. 6,216,706 to Kumar, which are incorporated herein by reference).

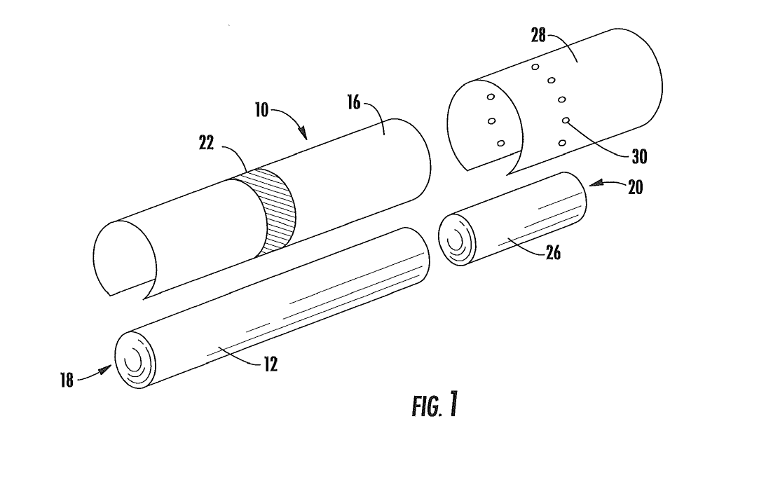

Referring to FIG. 1, there is shown a smoking article 10 in the form of a cigarette, wherein treated plant seeds as disclosed herein can be incorporated within one or more of the sections discussed hereinafter. The cigarette 10 includes a generally cylindrical rod 12 of a charge or roll of smokable filler material (e.g., about 0.3 to about 1.0 g of smokable filler material such as tobacco material treated as described herein) contained in a circumscribing wrapping material 16. The rod 12 is conventionally referred to as a “tobacco rod.” The ends of the tobacco rod 12 are open to expose the smokable filler material. The cigarette 10 is shown as having one optional band 22 (e.g., a printed coating including a film-forming agent, such as starch, ethylcellulose, or sodium alginate) applied to the wrapping material 16, and that band circumscribes the cigarette rod in a direction transverse to the longitudinal axis of the cigarette. That is, the band 22 provides a cross-directional region relative to the longitudinal axis of the cigarette. The band 22 can be printed on the inner surface of the wrapping material (i.e., facing the smokable filler material), or less preferably, on the outer surface of the wrapping material. Although the cigarette can possess a wrapping material having one optional band, the cigarette also can possess wrapping material having further optional spaced bands numbering two, three, or more.

At one end of the tobacco rod 12 is the lighting end 18, and at the mouth end 20 is positioned a filter element 26. The filter element 26 is positioned adjacent to one end of the tobacco rod 12 such that the filter element and tobacco rod are axially aligned in an end-to-end relationship, preferably abutting one another. Filter element 26 may have a generally cylindrical shape, and the diameter thereof may be essentially equal to the diameter of the tobacco rod. The ends of the filter element 26 permit the passage of air and smoke therethrough.

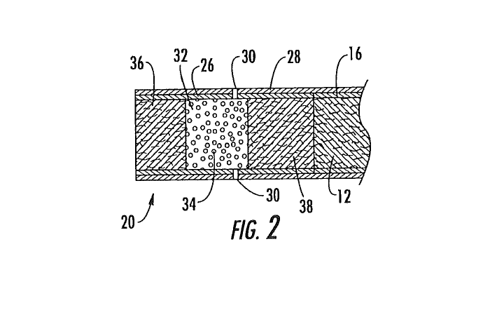

Treated plant seeds, as provided herein, can be incorporated within any one or more of the components of a smoking article as depicted (within a combustible portion or a non-combustible portion thereof). For example, treated plant seeds can be incorporated within the filter element (e.g., see FIG. 2), within the tobacco rod 12, or within the wrapping material. It is noted that, to facilitate inclusion of the plant seeds described herein within certain components of a smoking article, it may be beneficial to provide the seeds in a different form (i.e., not in substantially whole form). For example, treated plant seeds can be ground or otherwise processed to be dispersed within the tobacco rod, dispersed within the wrapping material, or adhered to the wrapping material.

A ventilated or air diluted smoking article can be provided with an optional air dilution means, such as a series of perforations 30, each of which extend through the plug wrap 28. The optional perforations 30 can be made by various techniques known to those of ordinary skill in the art, such as laser perforation techniques. Alternatively, so-called off-line air dilution techniques can be used (e.g., through the use of porous paper plug wrap and pre-perforated tipping paper). The filter element 26 is circumscribed along its outer circumference or longitudinal periphery by a layer of outer plug wrap 28, During use, the smoker lights the lighting end 18 of the cigarette 10 using a match or cigarette lighter. As such, the smokable material 12 begins to burn. The mouth end 20 of the cigarette 10 is placed in the lips of the smoker. Thermal decomposition products (e.g., components of tobacco smoke) generated by the burning smokable material 12 are drawn through the cigarette 10, through the filter element 26, and into the mouth of the smoker.

In some embodiments, the treated plant seed can be admixed with other components that are employed in the manufacture of tobacco products. Exemplary types of further ingredients that can be admixed with the treated plant seeds include flavorants, fillers, binders, pH adjusters, buffering agents, colorants, disintegration aids, antioxidants, humectants and preservatives. Representative tobacco blends, non-tobacco components, and representative cigarettes manufactured therefrom, are set forth in U.S. Pat. No. 4,836,224 to Lawson et al.; U.S. Pat. No. 4,924,888 to Perfetti et al.; U.S. Pat. No. 5,056,537 to Brown et al.; U.S. Pat. No. 5,220,930 to Gentry; and U.S. Pat. No. 5,360,023 to Blakley et al.; US Pat. Appl. Pub. No. 2002/0000235 to Shafer et al.; and PCT WO 02/37990. Those tobacco materials also can be employed for the manufacture of those types of cigarettes that are described in U.S. Pat. No. 4,793,365 to Sensabaugh; U.S. Pat. No. 4,917,128 to Clearman et al.; U.S. Pat. No. 4,947,974 to Brooks et al.; U.S. Pat. No. 4,961,438 to Korte; U.S. Pat. No. 4,920,990 to Lawrence et al.; U.S. Pat. No. 5,033,483 to Clearman et al.; U.S. Pat. No. 5,074,321 to Gentry et al.; U.S. Pat. No. 5,105,835 to Drewett et al.; U.S. Pat. No. 5,178,167 to Riggs et al.; U.S. Pat. No. 5,183,062 to Clearman et al.; U.S. Pat. No. 5,211,684 to Shannon et al.; U.S. Pat. No. 5,247,949 to Deevi et al.; U.S. Pat. No. 5,551,451 to Riggs et al.; U.S. Pat. No. 5,285,798 to Banerjee et al.; U.S. Pat. No. 5,593,792 to Farrier et al.; U.S. Pat. No. 5,595,577 to Bensalem et al.; U.S. Pat. No. 5,816,263 to Counts et al.; U.S. Pat. No. 5,819,751 to Barnes et al.; U.S. Pat. No. 6,095,153 to Beven et al.; U.S. Pat. No. 6,311,694 to Nichols et al.; and U.S. Pat. No. 6,367,481 to Nichols et al.; US Pat. Appl. Pub, No. 2008/0092912 to Robinson et al.; and PCT WO 97/48294 and PCT WO 98/16125. See, also, those types of commercially marketed cigarettes described Chemical and Biological Studies on New Cigarette Prototypes that Heat Instead of Burn Tobacco, R. J. Reynolds Tobacco Company Monograph (1988) and Inhalation Toxicology, 12:5, p. 1-58 (2000).

The treated plant seeds, or components thereof can, in some embodiments, be incorporated into a cigarette filter (e.g., in the filter plug, plug wrap, or tipping paper). For exemplary means by which a component such as a treated plant seed as disclosed herein can be incorporated into a cigarette filter, see US Pat. Appl. Pub. Nos. 2007/0186940 to Bhattacharyya et al, and 2008/0245376 to Travers et al., which are incorporated herein by reference. In a particular embodiment, the treated plant seeds are provided in at least one segment within a cigarette filter comprising multiple segments. For example, the treated plant seeds can be provided within a plug segment (e.g., dispersed within a fibrous tow) or can be provided within one or more cavities within a cigarette filter.

One exemplary embodiment of a filter element incorporating treated plant seeds according to the present disclosure, which is adapted for use with smoking articles such as cigarettes, is illustrated in FIG. 2. The filter element typically comprises multiple longitudinally extending segments. Each segment can have varying properties and may include various materials capable of filtration or adsorption of particulate matter and/or vapor phase compounds. Typically, the filter element of the invention includes 2 to 6 segments, frequently 2 to 4 segments. In one embodiment, the filter element includes a mouth end segment, a tobacco end segment and a compartment therebetween. This filter arrangement is sometimes referred to as a “compartment filter” or a “plug/space/plug” filter. The compartment may be divided into two or more compartments as described in greater detail below.

In FIG. 2, the illustrated filter element 20 comprises a first section of filter material 36, such as a fibrous filter material (e.g., plasticize cellulose acetate tow) and a second section of filter material 38 spaced apart from the first section of filter material. As shown, the first section of filter material 36 is positioned at the mouth end of the filter element 20 and the second section of filter material 38 is positioned proximal to the tobacco rod 12. The space between the first section of filter material 36 and the second section of filter material 38 define a compartment 32. At least a portion of the compartment 32 can contain treated plant seeds 34, as disclosed herein. Typically, in such embodiments, substantially the entire compartment 32 contains such treated plant seeds 34. However, it is also considered that the treated plant seeds may be admixed with one or more other materials within the compartment (e.g., adsorbents, such as activated carbon). However, the treated plant seed 34 could also be imbedded or dispersed within a section of filter material, such as a fibrous filter material (e.g., cellulose acetate tow), or incorporated into a paper. In addition, treated plant seeds 34 could be placed both in a compartment and imbedded in one or more of the sections of filter material, and the treated plant seeds in the compartment and the treated plant seeds imbedded or dispersed in the filter material can be the same or different.

Various filter element arrangements could be used without departing from the invention. The filter element of the invention typically comprises multiple, longitudinally-extending segments. Each segment can have varying properties and may include various materials capable of filtration or adsorption of particulate matter and/or vapor phase compounds. The filter element can further include a cavity formed between two filter tow segments. One or more sections of fibrous tow can also include channels or tubes formed therein.

Representative types of filter rods incorporating objects, and representative types of cigarettes possessing filter elements incorporating objects such as the treated plant seeds disclosed herein, can possess the types of components, format and configuration, and can be manufactured using the types of techniques and equipment set forth in U.S. Patent Application Publication Nos. 2011/0162665 to Burov et al., 2011/0162662 to Nikolov et al., 2008/0029118 to Nelson et al., 2006/0174899 to Luan et al., 2006/0130861 to Luan et al., 2005/0268925 to Schluter et al., 2004/0237984 to Figlar et al., and U.S. Pat. No. 7,972,254 to Stokes et al., U.S. Pat. No. 7,836,895 to Dube et al., U.S. Pat. No. 7,833,146 to Deal, U.S. Pat. No. 7,669,604 to Crooks et al., U.S. Pat. No. 7,479,098 to Thomas et al., U.S. Pat. No. 7,115,085 to Deal, U.S. Pat. No. 6,041,790 to Smith et al., U.S. Pat. No. 5,387,285 to Rivers, U.S. Pat. No. 4,862,905 to Green, Jr. et al., and U.S. Pat. No. 7,479,098 to Thomas et al.; which are incorporated herein by reference in their entireties.

Various types of cigarette components, including tobacco types, tobacco blends, top dressing and casing materials, blend packing densities; types of paper wrapping materials for tobacco rods, types of tipping materials, and levels of air dilution, can be employed. See, for example, the various representative types of cigarette components, as well as the various cigarette designs, formats, configurations and characteristics, which are set forth in U.S. Pat. No. 5,220,930 to Gentry, U.S. Pat. No. 6,779,530 to Kraker, and U.S. Pat. No. 7,237,559 to Ashcraft et al.; and US Pat. Appl. Pub, Nos. 2005/0066986 to Nestor et al., 2006/0272655 to Thomas et al., and 2007/0246055 to Oglesby; each of which is incorporated herein by reference.

The production of filter rods, filter rod segments and filter elements, and the manufacture of cigarettes from those filter rods, filter rod segments and filter elements, can be carried out using the types of equipment known in the art for such uses. Multi-segment cigarette filter rods can be manufactured using a cigarette filter rod making device available under the brand name Mulfi from Hauni-Werke Korber & Co. KG. Six-up rods, four-up filter rods and two-up rods that are conventionally used for the manufacture of filtered cigarettes can be handled using conventional-type or suitably modified cigarette rod handling devices, such as tipping devices available as Lab MAX, MAX, MAX S or MAX 80 from Hauni-Werke Korber & Co. KG. See, for example, the types of devices set forth in U.S. Pat. No. 3,308,600 to Erdmann et al.; U.S. Pat. No. 4,281,670 to Heitmann et al.; U.S. Pat. No. 4,280,187 to Reuland et al.; and U.S. Pat. No. 6,229,115 to Vos et al.

An exemplary apparatus to achieve the incorporation of treated plant seeds within a filter material may incorporate equipment for supplying a continuous supply of filter material to form a continuous filter rod (e.g., a filter tow processing unit adapted to supply filter tow to a continuous rod forming unit). A representative apparatus may also at least partially incorporate, for example, a rotating wheel arrangement such as disclosed in U.S. Pat. No. 7,479,098 to Thomas et al. and US Pat. Appl. Pub. No. US 2008/0302373 to Stokes et al., each of which is incorporated herein by reference. A representative apparatus can also include a feeder device for delivering the treated plant seeds to a rotating wheel insertion arrangement for insertion of the treated plant seeds into the filter material forming the continuous filter rod.

Filter elements produced in accordance with this disclosure may be incorporated within conventional cigarettes configured for combustion of a smokable material, and also within the types of cigarettes set forth in U.S. Pat. No. 4,756,318 to Clearman et al.; U.S. Pat. No. 4,714,082 to Banerjee et al.; U.S. Pat. No. 4,771,795 to White et al.; U.S. Pat. No. 4,793,365 to Sensabaugh et al.; U.S. Pat. No. 4,989,619 to Clearman et al.; U.S. Pat. No. 4,917,128 to Clearman et al.; U.S. Pat. No. 4,961,438 to Korte; U.S. Pat. No. 4,966,171 to Serrano et al.; U.S. Pat. No. 4,969,476 to Bale et al.; U.S. Pat. No. 4,991,606 to Serrano et al.; U.S. Pat. No. 5,020,548 to Farrier et al.; U.S. Pat. No. 5,027,836 to Shannon et al.; U.S. Pat. No. 5,033,483 to Clearman et al.; U.S. Pat. No. 5,040,551 to Schlatter et al.; U.S. Pat. No. 5,050,621 to Creighton et al.; U.S. Pat. No. 5,052,413 to Baker et al.; U.S. Pat. No. 5,065,776 to Lawson; U.S. Pat. No. 5,076,296 to Nystrom et al.; U.S. Pat. No. 5,076,297 to Farrier et al.; U.S. Pat. No. 5,099,861 to Clearman et al.; U.S. Pat. No. 5,105,835 to Drewett et al.; U.S. Pat. No. 5,105,837 to Barnes et al.; U.S. Pat. No. 5,115,820 to Hauser et al.; U.S. Pat. No. 5,148,821 to Best et al.; U.S. Pat. No. 5,159,940 to Hayward et al.; U.S. Pat. No. 5,178,167 to Riggs et al.; U.S. Pat. No. 5,183,062 to Clearman et al.; U.S. Pat. No. 5,211,684 to Shannon et al.; U.S. Pat. No. 5,240,014 to Deevi et al.; U.S. Pat. No. 5,240,016 to Nichols et al.; U.S. Pat. No. 5,345,955 to Clearman et al.; U.S. Pat. No. 5,396,911 to Casey, I I I et al.; U.S. Pat. No. 5,551,451 to Riggs et al.; U.S. Pat. No. 5,595,577 to Bensalem et al.; U.S. Pat. No. 5,727,571 to Meiring et al.; U.S. Pat. No. 5,819,751 to Barnes et al.; U.S. Pat. No. 6,089,857 to Matsuura et al.; U.S. Pat. No. 6,095,152 to Beven et al; and U.S. Pat. No. 6,578,584 to Beven; which are incorporated herein by reference.

The filter elements of the present invention can also be incorporated within aerosol-generating smoking articles that do not combust tobacco material to any significant degree, such as those set forth in U.S. Pat. No. 4,756,318 to Clearman et al.; U.S. Pat. No. 4,714,082 to Banerjee et al.; U.S. Pat. No. 4,771,795 to White et al.; U.S. Pat. No. 4,793,365 to Sensabaugh et al.; U.S. Pat. No. 4,989,619 to Clearman et al.; U.S. Pat. No. 4,917,128 to Clearman et al.; U.S. Pat. No. 4,961,438 to Korte; U.S. Pat. No. 4,966,171 to Serrano et al.; U.S. Pat. No. 4,969,476 to Bale et al.; U.S. Pat. No. 4,991,606 to Serrano et al.; U.S. Pat. No. 5,020,548 to Farrier et al.; U.S. Pat. No. 5,027,836 to Shannon et al.; U.S. Pat. No. 5,033,483 to Clearman et al.; U.S. Pat. No. 5,040,551 to Schlatter et al.; U.S. Pat. No. 5,050,621 to Creighton et al.; U.S. Pat. No. 5,052,413 to Baker et al.; U.S. Pat. No. 5,065,776 to Lawson; U.S. Pat. No. 5,076,296 to Nystrom et al.; U.S. Pat. No. 5,076,297 to Farrier et al.; U.S. Pat. No. 5,099,861 to Clearman et al.; U.S. Pat. No. 5,105,835 to Drewett et al.; U.S. Pat. No. 5,105,837 to Barnes et al.; U.S. Pat. No. 5,115,820 to Hauser et al.; U.S. Pat. No. 5,148,821 to Best et al.; U.S. Pat. No. 5,159,940 to Hayward et al.; U.S. Pat. No. 5,178,167 to Riggs et al.; U.S. Pat. No. 5,183,062 to Clearman et al.; U.S. Pat. No. 5,211,684 to Shannon et al.; U.S. Pat. No. 5,240,014 to Deevi et al.; U.S. Pat. No. 5,240,016 to Nichols et al.; U.S. Pat. No. 5,345,955 to Clearman et al.; U.S. Pat. No. 5,396,911 to Casey, I I I et al.; U.S. Pat. No. 5,551,451 to Riggs et al.; U.S. Pat. No. 5,595,577 to Bensalem et al.; U.S. Pat. No. 5,727,571 to Meiring et al.; U.S. Pat. No. 5,819,751 to Barnes et al.; U.S. Pat. No. 6,089,857 to Matsuura et al.; U.S. Pat. No. 6,095,152 to Beven et al; and U.S. Pat. No. 6,578,584 to Beven; and US Pat. Appl. Pub. Nos. 2010/0186757 to Crooks et al. and 2011/0041861 to Sebastian et al., which are incorporated herein by reference. Filter elements produced in accordance with the description provided above may be incorporated within “heat not burn” types of cigarettes that have been commercially marketed under the brand names “Premier” and “Eclipse” by R. J. Reynolds Tobacco Company. See, for example, those types of cigarettes described in Chemical and Biological Studies on New Cigarette Prototypes that Heat Instead of Burn Tobacco, R. J. Reynolds Tobacco Company Monograph (1988) and Inhalation Toxicology, 12:5, p. 1-58 (2000); which are incorporated herein by reference. Other examples of non-traditional cigarettes, commonly referred to as “e-cigarettes”, which could incorporate a filter element of the present invention, include U.S. Pat. No. 7,726,320 to Robinson et al. and U.S. Pat. No. 8,079,371 to Robinson et al., and US Pat. Appl. Publ. Nos. 2013/0037041 to Worm et al., 2013/0255702 to Griffith Jr. et al., and 2014/0000638 to Sebastian et al., all of which are incorporated by reference herein.

In practice, in one embodiment of a smoking article of the invention, smoke and/or vapor passes through a filter element, and comes into contact with the treated plant seeds disclosed herein. The modifying ingredient associated with the treated plant seeds is advantageously released from the seeds; the filtered smoke, altered in some fashion by the presence of the modifying ingredient, is then delivered to the user. The incorporation of a treated plant seed within a filter in this fashion can produce a smoking article which provides the smoker with mainstream smoke having altered chemical or sensory properties (e.g., altered flavor and/or aroma). Release of the modifying ingredient from the treated plant seed can typically be achieved under typical conditions of use of the smoking article.

In some embodiments, the modifying ingredient is released when the seed coat of the treated plant seed undergoes some type of physical destruction, breakage, or other loss of physical integrity (e.g., through disintegration, softening, fracture (e.g., microfracture), crushing, or the like). For example, in some embodiments, treated seeds within some portion of a tobacco product may be purposefully crushed by application of pressure to release the modifying ingredient (e.g., applied by hand (such as squeezing a filter containing such plant seeds) or applied after the product is inserted into the oral cavity (e.g., such as applying pressure by the tongue or teeth)).

In some embodiments, the treated plant seeds provide for passive release of the modifying ingredients associated therewith (i.e., the user does not need to exert pressure on the treated seed-containing portion of the smoking article to obtain release of at least some of the modifying ingredient(s) from the treated seeds). In such embodiments, the modifying ingredient associated therewith is passively released from the treated seed (e.g., through the intact seed coat or through fractures, e.g., microfractures preexisting in the seed coat).

In the context of smoking articles, the amount of treated plant seeds incorporated within a given article can vary, but will generally not exceed about 10 weight percent and will typically not exceed about 10 weight percent, based on the total dry weight of the smoking article within which the treated plant seed is incorporated. The amount of treated plant seeds incorporated can generally be the amount of treated plant seeds needed for the desired amount of modifying agent to be provided. This value will depend, in part, on the amount of modifying agent associated with each treated seed and the efficiency with which the modifying agent can be released from the seed. When the treated plant seed is employed within a smoking article, the amount of seed incorporated typically is at least about 0.5 weight percent, generally at least about 1 weight percent or at least about 2 weight percent, and often at least about 5 weight percent, based on the total dry weight of the smoking article. Typically, such values are less than about 25 weight percent, generally less than about 20 percent, and often less than about 15 percent, based on the total dry weight of the smoking article.

In some embodiments, the amount of treated seed incorporated within a smoking article can be calculated based on the desired amount of modifying agent to be incorporated. For example, where the modifying agent to be incorporated is menthol, a typical amount of menthol incorporated within a smoking article is between about 1 and about 10 mg per smoking article. Accordingly, in such embodiments, plant seeds treated with menthol can be analyzed to determine the amount of menthol associated with each seed and the requisite weight of treated plant seeds to obtain the desired amount of menthol can be calculated. As would be readily apparent, the above amounts would also be useful for other modifying ingredients such as other flavorants.

It is understood that the amount of modifying ingredient associated with a given type of treated plant seed may impact these values, as a higher content of treated seeds may be used where less of the modifying ingredient (e.g., a diluted sample of modifying ingredient) is associated with the treated seeds. The specific properties of the plant seeds themselves may also impact these values. For example, one would expect that seeds capable of retaining and/or releasing lesser amounts of modifying ingredient may require a higher amount of seeds to achieve a comparable release of the modifying ingredient.

The treated plant seeds described herein can also be incorporated into smokeless tobacco products, such as loose moist snuff (e.g., snus), loose dry snuff, chewing tobacco, pelletized tobacco pieces (e.g., having the shapes of pills, tablets, spheres, coins, beads, obloids or beans), extruded or formed tobacco strips, pieces, rods, cylinders or sticks, finely divided ground powders, finely divided or milled agglomerates of powdered pieces and components, flake-like pieces, molded processed tobacco pieces, pieces of tobacco-containing gum, rolls of tape-like films, readily water-dissolvable or water-dispersible films or strips (e.g., US Pat. App. Pub. No. 2006/0198873 to Chan et al.), or capsule-like materials possessing an outer shell (e.g., a pliable or hard outer shell that can be clear, colorless, translucent or highly colored in nature) and an inner region possessing tobacco or tobacco flavor (e.g., a Newtonian fluid or a thixotropic fluid incorporating tobacco of some form). Various types of smokeless tobacco products are set forth in U.S. Pat. No. 1,376,586 to Schwartz; U.S. Pat. No. 3,696,917 to Levi; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,987,907 to Townsend; U.S. Pat. No. 5,092,352 to Sprinkle, I I I et al.; and U.S. Pat. No. 5,387,416 to White et al.; US Pat. App. Pub. Nos. 2005/0244521 to Strickland et al. and 2008/0196730 to Engstrom et al.; PCT WO 04/095959 to Arnarp et al.; PCT WO 05/063060 to Atchley et al.; PCT WO 05/016036 to Bjorkholm; and PCT WO 05/041699 to Quinter et al., each of which is incorporated herein by reference. See also, the types of smokeless tobacco formulations, ingredients, and processing methodologies set forth in U.S. Pat. No. 6,953,040 to Atchley et al.; U.S. Pat. No. 7,032,601 to Atchley et al.; and U.S. Pat. No. 7,810,507 to Dube et al.; US Pat. Appl. Pub. Nos. 2002/0162562 to Williams; 2002/0162563 to Williams; 2003/0070687 to Atchley et al.; 2004/0020503 to Williams, 2005/0178398 to Breslin et al.; 2006/0191548 to Strickland et al.; 2007/0062549 to Holton, Jr. et al.; 2007/0186941 to Holton, Jr. et al.; 2007/0186942 to Strickland et al.; 2008/0029116 to Robinson et al.; 2008/0029117 to Mua et al.; 2008/0173317 to Robinson et al.; 2008/0209586 to Neilsen et al.; 2010/0018541 to Gerardi et al.; 2010/0018540 to Doolittle et al.; and 2010/0116281 to Marshall et al., each of which is incorporated herein by reference.

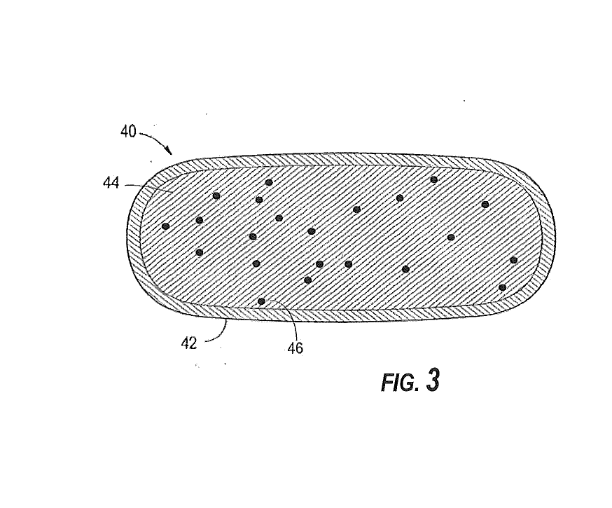

Referring to FIG. 3, a representative snus type of tobacco product comprising the syrup of the present invention is shown. In particular, FIG. 3 illustrates a smokeless tobacco product 40 having a water-permeable outer pouch 42 containing a smokeless tobacco composition 44, wherein the tobacco composition includes a plurality of treated plant seeds 46 according to the present disclosure. The outer pouch 42 can be the type commonly used for the manufacture of snus types of products (e.g., a sealed, moisture permeable pouch that is sometimes referred to as a “portion”). A representative moisture permeable pouch can be composed of a “fleece” type of material.

In one embodiment, a plurality of the treated plant seeds described herein may be incorporated within the outer pouch along with the tobacco formulation as shown in FIG. 3. During use of the product within the oral cavity, at least a portion of the modifying ingredient(s) associated with the treated plant seeds can be released from the seed under typical conditions of use. In some embodiments, the modifying ingredient(s) can be released from the treated seeds by moistening of the product within the oral cavity. In some embodiments, crushing or other physical destruction of the treated plant seeds can provide release of the modifying ingredient from the treated seeds. The modifying ingredient is generally capable of dissolving in saliva such that, when released from the treated seeds, the modifying agent(s) can pass through the pouch material and into the oral cavity. Release of the modifying agent(s) in this manner can provide various functional benefits such enhanced taste (with the specific benefit(s) afforded depending on the composition of the modifying ingredient(s) associated with the treated plant seeds, as described in detail herein).

The number of treated plant seeds incorporated into a smokeless tobacco product can vary, depending upon factors such as the size of the treated seeds, the character or nature of the modifying ingredient associated therewith, the desired attributes of the smokeless tobacco product, and the like. The number of treated plant seeds incorporated within smokeless tobacco product can exceed about 5, can exceed about 10, can exceed about 20, can exceed about 40, and can even exceed about 100. In certain embodiments, the number of treated plant seeds can be greater than about 500, and even greater than about 1,000. Within a smokeless tobacco product, the amount of treated plant seed typically is at least about 5 ppm, generally at least about 10 ppm, and often at least about 100 ppm, based on the total dry weight of the tobacco material within the smokeless tobacco product; but typically is less than about 25 percent, generally less than about 10 percent, 5 percent, or 2 percent, and often less than about 1 percent, based on the total dry weight of the tobacco material within the smokeless tobacco product.

Further ingredients can be admixed with, or otherwise incorporated within, a smokeless tobacco compositions according to the present disclosure. The additives can be artificial, or can be obtained or derived from herbal or biological sources. Exemplary types of additives include salts (e.g., sodium chloride, potassium chloride, sodium citrate, potassium citrate, sodium acetate, potassium acetate, and the like), natural sweeteners (e.g., fructose, sucrose, glucose, maltose, vanillin, ethylvanillin glucoside, mannose, galactose, lactose, and the like), artificial sweeteners (e.g., sucralose, saccharin, aspartame, acesulfame K, neotame and the like), organic and inorganic fillers (e.g., grains, processed grains, puffed grains, maltodextrin, dextrose, calcium carbonate, calcium phosphate, corn starch, lactose, manitol, xylitol, sorbitol, finely divided cellulose, and the like), binders (e.g., povidone, sodium carboxymethylcellulose and other modified cellulosic types of binders, sodium alginate, xanthan gum, starch-based binders, gum arabic, lecithin, and the like), pH adjusters or buffering agents (e.g., metal hydroxides, preferably alkali metal hydroxides such as sodium hydroxide and potassium hydroxide, and other alkali metal buffers such as metal carbonates, preferably potassium carbonate or sodium carbonate, or metal bicarbonates such as sodium bicarbonate, and the like), colorants (e.g., dyes and pigments, including caramel coloring and titanium dioxide, and the like), humectants (e.g., glycerin, propylene glycol, and the like), oral care additives (e.g., thyme oil, eucalyptus oil, and zinc), preservatives (e.g., potassium sorbate, and the like), syrups (e.g., honey, high fructose corn syrup, and the like), disintegration aids (e.g., microcrystalline cellulose, croscarmellose sodium, crospovidone, sodium starch glycolate, pregelatinized corn starch, and the like), flavorant and flavoring mixtures, antioxidants, and mixtures thereof. If desired, the additive can be microencapsulated as set forth in U.S. Pat. No. 7,810,507 to Dube et al., which is incorporated by reference herein. In addition, exemplary encapsulated additives are described, for example, in WO 2010/132444 A2 to Atchley, which has been previously incorporated by reference herein.

The smokeless tobacco product of the invention can take various forms, including forms that do not require an outer pouch or fleece, such as smokeless products in the form of a dissolvable or meltable formulation. The smokeless tobacco compositions of the invention can include various additional components, such as a water-soluble polymeric binder material and optionally other ingredients that provide a dissolvable composition that will slowly disintegrate in the oral cavity during use. In certain embodiments, the smokeless tobacco composition can include lipid components that provide a meltable composition that melts (as opposed to merely dissolving) in the oral cavity, such as compositions set forth in US Appl. Pub. No. 2012/0037175 to Cantrell et al., which is incorporated by reference herein.

In further embodiments, treated plant seeds as disclosed herein can be provided in individual seed form. For example, plant seeds can be treated as provided according to the present disclosure and can be directly placed in the oral cavity in seed form (i.e., not within any matrix). For example, such seeds can be provided in the absence of a tobacco composition. A single seed can be placed in the oral cavity or a plurality of seeds can be placed in the oral cavity (optionally encased in a pouch or fleece as shown in FIG. 3). In such embodiments, the modifying ingredient can be released from the treated seeds passively (e.g., wherein the modifying ingredient is released from the treated seeds based on the moistening conditions within the oral cavity, such as where the moistening conditions soften the seed coat sufficiently to allow the modifying ingredient to penetrate therethrough) and/or actively (e.g., wherein the modifying ingredient is released from the treated seeds by fracture, crushing, or the like, which can be accomplished, e.g., by applying pressure by the tongue or teeth). Although the modifying ingredient in such independent seed forms can be any of the types of ingredients described herein, in certain embodiments, the modifying ingredient in such forms comprises a tobacco extract, such as an aqueous tobacco extract (including pharmaceutical grade nicotine), and in such embodiments, can also be viewed as a tobacco product even where the seed is not from a Nicotiana species.

Representative tobacco extracts include, but are not limited to, those types of extracts described and/or referenced in U.S. Pat. No. 5,159,942 to Brinkley et al., and US Pat. Appl. Pub. Nos. 2011/0259353 to Coleman, I I I et al., 2012/0125354 to Byrd, Jr. et al., 2012/0211016 to Byrd, Jr. et al., 2012/0272976 to Byrd, Jr. et al., 2012/0291793 to Byrd, Jr. et al., 2013/0074856 to Holton et al., 2014/0096780 to Gerardi et al., and 2014/0271951 to Mua et al., which are incorporated by reference herein.

### Experimental Example

The present invention is more fully illustrated by the following example, which is set forth to illustrate the present invention and is not to be construed as limiting thereof. Unless otherwise noted, all parts and percentages are by weight, and all weight percentages are expressed on a dry basis, meaning excluding water content, unless otherwise indicated.

Tobacco seeds (9.3 g of flue cured tobacco seeds) are incubated at 37° C. for 4 weeks in 35 mL of medium chain triglycerides (MTC) containing 30% menthol by volume. The seeds are washed by placing them on sieve screens and washing with cold water to remove residual oils. The seeds are air dried overnight and are further evaluated. To evaluate the menthol concentration, approximately 0.5 g of seeds is weighed and recorded. To the seeds is added 20 mL methanol with trans anethol added as an internal standard and the sample is shaken in a heated stir bath at 250 RPM and 55° C. for one hour. An aliquot of the sample is transferred to a vial and analyzed by gas chromatography (GC 2 a 6890 GC with Split/Split less injector and FID detector). The concentration from the GC is reported in EZChrom Open Lab and transferred to LIMS (LIMS calculated GC concentration*20*100/wt./1000). Based on this method, the menthol concentration of the treated seeds was approximately 3% by volume.

The treated seeds are then introduced into a smoking article to evaluate menthol delivery therefrom. Treated seeds (˜100-110 mg) are added to each of four cavities within a 108 mm 4 up rod cigarette filter. Accordingly, the 4 up rod cigarette filter contains about 400 mg of seed, distributed among four cavities within the filter. The 4 up rod cigarette filter is segmented by splitting the rod into eight (27 mm) segments. The 27 mm segment is associated with a cut filler tobacco rod, tipping paper is applied, and the resulting smoking article is sealed to provide a prototype cigarette comprising a seed-containing filter component.

An informal sensory evaluation is conducted to assess the menthol delivery when the prototype cigarette is smoked. This evaluation confirmed that some degree of menthol (at a relatively low level) is introduced into the mainstream smoke of the cigarette, as detected by the smoker.

We claim:

1. A tobacco product comprising a tobacco formulation and at least one treated plant seed or portion thereof carrying one or more modifying ingredients.

2. The tobacco product of claim 1, wherein the treated plant seed or portion thereof comprises a seed or portion thereof from the Nicotiana species.

3. The tobacco product of claim 1, wherein the treated plant seed or portion thereof comprises an edible seed or portion thereof.

4. The tobacco product of claim 1, wherein the treated plant seed or portion thereof comprises a seed selected from the group consisting of tobacco seeds, poppy seeds, sesame seeds, sunflower seeds, pumpkin seeds, chia seeds, flax seeds, hemp seeds, papaya seeds, cocoa seeds, and soybean seeds.

5. The tobacco product of claim 1, wherein the one or more modifying ingredients are selected from the group consisting of water, flavorants, sweeteners, colorants, pH adjusters, buffering agents, oral care additives, humectants, antioxidants, preservatives, additives derived from herbal or botanical sources, and mixtures thereof.

6. The tobacco product of claim 1, wherein the one or more modifying ingredients comprise a flavorant.

7. The tobacco product of claim 6, wherein the flavorant imparts a flavor selected from the group consisting of vanilla, coffee, chocolate, cream, mint, spearmint, eucalyptus, menthol, peppermint, wintergreen, lavender, cardamom, nutmeg, cinnamon, clove, cascarilla, sandalwood, honey, jasmine, ginger, anise, sage, licorice, lemon, orange, apple, peach, lime, cherry, strawberry, and combinations thereof.

8. The tobacco product of claim 6, wherein the flavorant comprises menthol.

9. The tobacco product of claim 1, wherein the tobacco product is in the form of a smoking article, wherein the one or more modifying ingredients are adapted for release into mainstream smoke generated by the smoking article.

10. The tobacco product of claim 9, wherein the smoking article comprises a filter element comprising the at least one treated plant seed or portion thereof.

11. The tobacco product of claim 10, wherein the at least one treated plant seed or portion thereof is positioned within a cavity within the filter element or dispersed within a fibrous tow segment.

12. The tobacco product of claim 1, wherein the tobacco product is in the form of a smokeless tobacco composition, wherein the one or more modifying ingredients are adapted for release in the oral cavity.

13. The tobacco product of claim 1, wherein the treated plant seed or portion further comprises one or more diluting agents associated therewith.

14. The tobacco product of claim 13, wherein the one or more diluting agents comprise triglycerides.

15. A method for modifying the properties of a tobacco product, comprising:
i) receiving a treated plant seed or portion thereof carrying one or more modifying ingredients; and
ii) adding the treated plant seed to the tobacco product.

16. The method of claim 15, wherein the treated plant seed or portion thereof comprises a seed or portion thereof from the Nicotiana species.

17. The method of claim 15, wherein the treated plant seed or portion thereof comprises an edible seed or portion thereof.

18. The method of claim 15, wherein the treated plant seed or portion thereof comprises a seed selected from the group consisting of tobacco seeds, poppy seeds, sesame seeds, sunflower seeds, pumpkin seeds, chia seeds, flax seeds, hemp seeds, papaya seeds, cocoa seeds, and soybean seeds.

19. The method of claim 15, wherein the one or more modifying ingredients are selected from the group consisting of water, flavorants, sweeteners, colorants, pH adjusters, buffering agents, oral care additives, humectants, antioxidants, preservatives, additives derived from herbal or botanical sources, and mixtures thereof.

20. The method of claim 15, wherein the one or more modifying ingredients comprise a flavorant.

21. The method of claim 20, wherein the flavorant imparts a flavor selected from the group consisting of vanilla, coffee, chocolate, cream, mint, spearmint, eucalyptus, menthol, peppermint, wintergreen, lavender, cardamom, nutmeg, cinnamon, clove, cascarilla, sandalwood, honey, jasmine, ginger, anise, sage, licorice, lemon, orange, apple, peach, lime, cherry, strawberry, and combinations thereof.

22. The method of claim 20, wherein the flavorant comprises menthol.

23. The method of claim 15, wherein the tobacco product is in the form of a smoking article comprising a filter element and the adding step comprises incorporating the treated plant seed or portion thereof within the filter element.

24. The method of claim 23, wherein the filter element comprises at least one cavity or at least one segment of fibrous tow, and the adding step comprises incorporating the treated plant seed or portion thereof within the at least one cavity or the at least one segment of fibrous tow.

25. The method of claim 15, further comprising treating a harvested plant seed or portion thereof by contacting the plant seed or portion thereof with a modifying ingredient to give a treated plant seed or portion thereof.

26. The method of claim 25, wherein the treating step comprises immersing the harvested plant seed or portion thereof in a liquid comprising the modifying ingredient.

27. The method of claim 26, wherein the liquid further comprises one or more solvents.

28. The method of claim 25, wherein the treating step is conducted at one or both of elevated temperature and elevated pressure.

29. A method for modifying the properties of a tobacco product, comprising:
i) treating a harvested plant seed or portion thereof by contacting the plant seed or portion thereof with a modifying ingredient to give a treated plant seed or portion thereof; and
ii) adding the treated plant seed or portion thereof to a tobacco product.

30. The method of claim 29, wherein the treated plant seed or portion thereof comprises a seed or portion thereof from the Nicotiana species.

31. The method of claim 29, wherein the treating step comprises immersing the harvested plant seed or portion thereof in a liquid comprising the modifying ingredient.

32. The method of claim 31, wherein the liquid further comprises one or more solvents.

33. The method of claim 29, wherein the treating step is conducted at one or both of elevated temperature and elevated pressure.

34. The method of claim 29, wherein the tobacco product is in the form of a smoking article comprising a filter element and the adding step comprises incorporating the treated plant seed or portion thereof within the filter element.

35. The method of claim 29, further comprising the step of pre-treating the harvested plant seed or portion thereof having a seed coat to soften the seed coat or to create microfractures in the seed coat in order to enhance penetration of the modifying ingredient therethrough.

36. A tobacco product in the form of a seed or portion thereof from the Nicotiana species carrying a modifying ingredient infused therein.

37. The tobacco product of claim 36, wherein the modifying ingredient is a flavorant.
